(19) United States
(12) Patent Application Publication
(10) Pub. No.: US 2014/0099685 A1
(43) Pub. Date: Apr. 10, 2014

(54) BIOREACTORS APPARATUS, SYSTEM AND METHOD

(71) Applicant: JOULE UNLIMITED TECHNOLOGIES, IN., Bedford, MA (US)

(72) Inventors: Max B. Tuttman, Somerville, MA (US); Stuart A. Jacobson, Lexington, MA (US); Rainer Ponzel, Hoevelhof (DE); John E. Longan, Nashua, NH (US)

(21) Appl. No.: 14/122,139

(22) PCT Filed: May 24, 2012
(86) PCT No.: PCT/US2012/039366

§ 371 Date: Nov. 25, 2013

Related U.S. Application Data
(60) Provisional application No. 61/491,026, filed on May 27, 2011.

(51) Int. Cl. C12M 1/00; C12M 1/02

(52) U.S. Cl. CPC C12M 21/02; C12M 23/22; C12M 31/00; C12M 27/00
USPC 435/134; 435/292.1; 435/257.1; 435/161; 435/160; 435/157; 435/167

(57) ABSTRACT

The present invention provides a bioreactor having a reactor chamber and one or more support chambers. The reactor chamber can have one or more flexible walls for enclosing microorganisms and culture medium. The reactor chamber provides an enclosure for microorganism and culture medium. The support chamber can also have one or more flexible walls. When inflated to a predetermined amount, the support chamber causes the microorganisms and culture medium to distribute to a substantially even depth across the reactor chamber.

### RELATED APPLICATION(S)

This application claims the benefit of U.S. Provisional Application No. 61/491,026, filed on May 27, 2011. The entire teachings of the above application(s) are incorporated herein by reference.

### BACKGROUND OF THE INVENTION

One of the primary limitations of using photosynthetic microorganisms as a method of carbon dioxide sequestration or conversion to products has been the need for development of efficient and cost-effective growth systems. Aquatic organisms, such as algae, oysters, and lobsters, have been cultured primarily in open systems. This approach allows for the organisms to take advantage of the semi-natural environment while keeping operational expenditures potentially low. Open algal ponds up to 4 km² have been researched, which, while requiring low capital expenditures, ultimately have low productivity as these systems are also subject to a number of problems. Intrinsic to being an open system, the cultured organisms are exposed to a number of exogenous organisms that can be symbiotic, competitive, or pathogenic. Symbiotic organisms can change the culture organisms merely by exposing them to a different set of conditions. Opportunistic species can compete with the desired organism for space, nutrients, etc. Additionally, pathogenic invaders can feed on or kill the desired organism. In addition to these complicating factors, open systems are difficult to insulate from environmental changes including temperature, turbidity, pH, salinity, and exposure to the sun. These difficulties point to the need to develop a closed, controllable system for the growth of algae and similar organisms.

Not surprisingly, a number of closed photobioreactors have been developed. Typically, these are cylindrical or tubular (i.e., U.S. Pat. No. 5,958,761, U.S. Patent application No. 2007/0048859). These bioreactors often require mixing devices, increasing cost, and are prone to accumulating oxygen (O₂), which inhibits algal growth.

Tubular bioreactors, when oriented horizontally, typically require additional energy to provide mixing (e.g., pumps), thus adding significant capital and operational expense. In this orientation, the O₂ produced by photosynthesis can readily become trapped in the system, thus causing a significant reduction in organism proliferation.

Several flat-plate photobioreactor designs have been disclosed for culturing microalgae: Samson, R. & Leduy, A. (1985), Multistage continuous cultivation of blue-green alga Spinilina maxima in the flat tank photobioreactors with recycle. Can. J. Chem. Eng. 63: 105-1 12; Ramos de Ortega and Roux, J. C. (1986), Production of Chlorella biomass in different types of flat bioreactors in temperate zones. Biomass 10: 141-156; Tredici, M. R. and Materassi, R. (1992), From open ponds to vertical alveolar panels: the Italian experience in the development of reactors for the mass cultivation of photoautotrophic microorganisms. J. Appl. Phycol 4: 221-31; Tredici M. R., Carlozzi P., Zittelli G. C. and Materassi R. (1991), A vertical alveolar panel (VAP) for outdoor mass cultivation of microalgae and Cyanobacteria. Bioresource Technol. 38: 153-159; Hu Q. and Richmond A. (1996), Productivity and photosynthetic efficiency Spirulina platensis as affected by light intensity, organism density and rate of mixing in a flat plate photobioreactor. J. Appl. Phycol. 8: 139-145; Hu Q, Yair Z. and Richmond A. (1998) Combined effects of light intensity, light-path and culture density on output rate of Spirulina platensis (Cyanobacteria). European Journal of Phycology 33: 165-171; Hu et al. WO 2007/098150. However, to date, no design or system has been successfully scaled up for efficient growth of organisms in commercial scale.

Many different photobioreactor configurations have been described in the literature including flat panels, bubble columns, tubular reactors and a variety of annular designs aimed at improving the surface area to volume ratio to maximize conversion of sunlight and CO₂ to biomass or other products such as algal oil. These reactors have distinct advantages compared to open raceway with respect to controlling temperature, pH, nutrients and limiting contamination (see Pulz, O. “Photobioreactors: Production systems for phototrophic microorganisms,” Appl. Microbiol. Biotechnol (2001) 57:287-293). Key limitations to their adoption have been the cost vs. benefit as it relates to the product being produced. Whereas valuable products such as carotenoids have been produced in photobioreactors, the production of biomass for fuels has not been economically justified to date.

What is needed, therefore, is an integrated photobioreactor system that is scalable, low cost, and efficient for culturing light-capturing organisms.

### SUMMARY OF THE INVENTION

One embodiment of the present invention is a photobioreactor or bioreactor, system, or method thereof. The photobioreactor can include a reactor chamber and one or more support chambers. The reactor chamber can have one or more flexible walls for enclosing microorganisms and culture medium. The reactor chamber provides an enclosure for microorganism and culture medium. The support chamber can also have one or more flexible walls. When inflated to a predetermined amount, the support chamber causes the microorganisms and culture medium to distribute to a substantially even depth across the reactor chamber.

Other embodiments can include one or more of the following variations. The substantially even depth across the reactor chamber can average between about 5 mm to about 30 mm. In another embodiment, the predetermined amount can cause the microorganisms and culture medium to distribute to a substantially even depth across the reactor chamber in a convex, crescent shape. In another embodiment, the support chamber is a base chamber positioned underneath the reactor chamber and/or a cover chamber positioned overtop the reactor chamber. In another embodiment, the reactor can further include a thermal chamber in thermal contact with the reactor chamber. In another embodiment, a reactor capsule encloses the microorganisms and the culture medium and the reactor capsule can be housed within the reactor chamber. In another embodiment, inflating the support chamber to a predetermined amount can cause the microorganisms and culture medium to evacuate the reactor chamber. In another embodiment, the support chamber comprises multiple lateral chambers and/or multiple segments. In another embodiment, inflating one or more of each multiple lateral chambers and/or segments induces mixing the microorganisms and culture medium within the reactor chamber. In another embodiment, a first sheet, a second sheet, and a third sheet are connected lengthwise along opposing ends and the first sheet and the second sheet produce the flexible walls of the reactor chamber and the second sheet and the third sheet produce the flexible walls of the support chamber.

In yet another embodiment, inflating the support chamber to a predetermined amount causes a surface area of the microorganisms and culture medium to increase. In yet another embodiment, inflating the support chamber to a predetermined amount causes a surface area of the microorganisms and culture medium to increase and the depth of the microorganisms and culture medium to decrease.

A further embodiment of the present invention is a photobioreactor comprising: a first enclosure for containing a culture medium with a photoautotrophic microorganism and a second enclosure; wherein (1) the first enclosure and the second enclosure are layered with the second enclosure being below the first enclosure, (2) the first enclosure having at least a flexible bottom section and the second enclosure having at least a flexible top section, (3) the first enclosure and second enclosure are positioned such that controllably pressurizing the second enclosure controllably changes the shape of the first enclosure containing a culture medium with a phototrophic microorganism and a gas, and (4) the first enclosure being at least in part transparent to allow light of a wavelength that is photosynthetically active in the phototrophic microorganism to reach the culture medium.

The present invention is not intended to be limited to a system or method that must satisfy one or more of any stated objects or features of the invention. It is also important to note that the present invention is not limited to the exemplary or primary embodiments described herein. Modifications and substitutions by one of ordinary skill in the art are considered to be within the scope of the present invention.

### BRIEF DESCRIPTION OF THE DRAWINGS

These and other features and advantages of the present invention will be better understood by reading the following detailed description, taken together with the drawings wherein:

FIG. 1 is a profile block diagram of a photobioreactor constructed in accordance with an exemplary embodiment of the invention.

FIG. 2 is a profile diagram of a photobioreactor with a reactor chamber and support chambers constructed in accordance with an exemplary embodiment of the invention.

FIG. 3 is a perspective view of a photobioreactor with a reactor chamber and support chambers constructed in accordance with an exemplary embodiment of the invention.

FIG. 4 is a profile diagram of a photobioreactor with a reactor chamber, support chambers and thermal exchange chamber constructed in accordance with an exemplary embodiment of the invention.

FIG. 5 is a profile diagram of a photobioreactor with a reactor chamber with reactor capsule and support chambers constructed in accordance with an exemplary embodiment of the invention.

FIG. 6 is a profile diagram of a photobioreactor with reactor chamber with a reactor capsule within a thermal exchange chamber constructed in accordance with an exemplary embodiment of the invention.

FIG. 7 is a profile diagram of a photobioreactor with support chambers providing a crescent shaped reactor chamber constructed in accordance with an exemplary embodiment of the invention.

FIG. 8 is a profile diagram of a photobioreactor with a reactor chamber and multiple lateral support chambers constructed in accordance with an exemplary embodiment of the invention.

FIG. 9 is a perspective view of a photobioreactor with a reactor chamber and multiple lateral support chambers constructed in accordance with an exemplary embodiment of the invention.

FIGS. 10A-C are profile diagrams of a photobioreactor with a reactor chamber and multiple lateral support chambers constructed in accordance with an exemplary embodiment of the invention.

FIGS. 11A-C are profile diagrams of a photobioreactor with a reactor chamber and multiple segmented support chambers constructed in accordance with an exemplary embodiment of the invention.

FIG. 12 is a perspective view of a photobioreactor with a reactor chamber and support structures constructed in accordance with an exemplary embodiment of the invention.

The drawings are not necessarily to scale, emphasis instead being placed upon illustrating embodiments of the present invention.

### DETAILED DESCRIPTION OF THE INVENTION

A description of preferred embodiments of the invention follows. While this invention has been particularly shown and described with references to preferred embodiments thereof, it will be understood by those skilled in the art that various changes in form and details can be made therein without departing from the scope of the invention encompassed by the appended claims.

The following explanations of terms and methods are provided to better describe the present invention and to guide those of ordinary skill in the art in the practice of the present invention. As used herein, “comprising” means “including” and the singular forms “a” or “an” or “the” include plural references unless the context clearly dictates otherwise. For example, reference to “comprising a phototrophic microorganism” includes one or a plurality of such phototrophic microorganisms. The term “or” refers to a single element of stated alternative elements or a combination of two or more elements, unless the context clearly indicates otherwise.

Unless explained otherwise, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are illustrative only and not intended to be limiting. Other features of the invention are apparent from the following detailed description and the claims.

General

Exemplary embodiments of the invention can provide an inflatable reactor chamber for a photobioreactor to provide functions of culture containment, photon capture, temperature control, pH control, and CO2 injection in a highly integrated design, lowering overall manufacturing, material, and deployment costs. The exemplary embodiments of the invention can facilitate high volume manufacturing, ease of deployment and mass deployment with unprecedented scalability. The reactor chamber design can have several layers of thin film, sealed lengthwise in order to create layered chambers. These chambers can be pressurized with different fluids, including air, culture, thermal coolant fluid, and flue gas to create a balance of pressures that can shape the culture layer within the culture chamber. In particular, the pressure balance design can be used to produce a high aspect ratio culture layer, which is desired for high biological productivity. The presented embodiments can remove the need for more costly ways of maintaining thin culture layers, such as thicker materials, or more involved manufacturing processes like spot welding, thereby reducing the potential for leakage and increasing overall product reliability at scale.

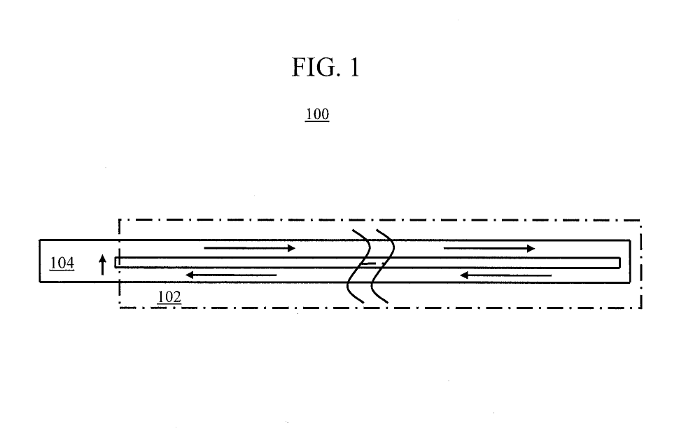

Referring to FIG. 1, an exemplary photobioreactor 100 includes a horizontally oriented reactor chamber 102 and a circulation driver 104 to provide circulation. The circulation driver 104 provides a flow of material in the thin-film photobioreactor, which can be made of translucent or transparent material. The reactor chamber 102 can be a thin-film chamber with a high aspect ratio (e.g, thin in cross-sectional view). The culture medium and organism can circulate through the reactor chamber 102 and maximize exposure via the increased high aspect ratio. After circulating through a loop of the reactor chamber 102, the culture medium and organism can exit into the circulation driver 104 and back into the entrance of the reactor chamber 102. The reactor chamber 102 can be designed in an elongated loop with a path extending away from the circulation driver 104 and returning via a return path parallel from the away path. Embodiments are not limited to one circulation driver or an enclosed loop as shown in FIG. 1. Exemplary embodiments can utilize multiple circulation drivers 104 and/or can comprise a single directional path.

Example circulation drivers 104 are not limited to utilizing an induced circulation system, for example, gravity driven, tidal driven, air driven, thermally driven, or circulation systems that do not involve active contact with the material being circulated. Exemplary circulating drivers 104 can also utilize active circulation devices such as pumps, augers, and conveyor belts that actively apply a contact and apply a force to the circulating material.

Exemplary embodiments of the invention can provide the reactor chamber 102 for culture containment and productions utilizing various aspects either individually or combined as discussed in the following exemplary embodiments.

A further embodiment of the present invention is a photobioreactor comprising: a first enclosure for containing a culture medium with a photoautotrophic microorganism and a second enclosure; wherein (1) the first enclosure and the second enclosure are layered with the second enclosure being below the first enclosure, (2) the first enclosure having at least a flexible bottom section and the second enclosure having at least a flexible top section, (3) the first enclosure and second enclosure are positioned such that controllably pressurizing the second enclosure controllably changes the shape of the first enclosure containing a culture medium with a phototrophic microorganism and a gas, and (4) the first enclosure being at least in part transparent to allow light of a wavelength that is photosynthetically active in the phototrophic microorganism to reach the culture medium.

The first enclosure can be a reactor chamber or a cover chamber as described herein, the second enclosure can be a base chamber as described herein, the third enclosure can be cover chamber as described herein, the fourth can be a heat exchange chamber as described herein, and the fifth enclosure can be a reactor capsule as described herein.

Typically, the first, second, third and fourth enclosures are formed from a thin-film polymer material. Each of the enclosures can be formed from one or more polymer sheets. Typically, the use of fewer polymer sheets in preparing an enclosure is desirable. Also, typically, fewer seals are preferable. Accordingly, more typically, the enclosures can be formed from two polymer sheets, wherein in the case of two directly adjacent enclosures, one polymer sheet (i.e., the one separating the enclosure volumes) can form part of both of the adjacent enclosures. Alternatively, thin-film polymer tubes can be used. Accordingly, two enclosures can be formed in a process including lengthwise sealing (e.g., with two seals) of at least three polymer sheets; three enclosures can be formed in a process including lengthwise sealing (e.g., with two seals) of at least four polymer sheets; and four enclosures can be formed in a process including lengthwise sealing (e.g., with two seals) of at least five polymer sheets. In the case of enclosures prepared from tubes and/or polymer sheets, two enclosures can be formed by sealing (e.g., with two seals) one polymer tube and one polymer sheet (placed inside the polymer tube); three enclosures can be formed by sealing (e.g., with two seals) a first polymer tube with a second polymer tube having a smaller radius placed (lengthwise) within the first polymer tube; and four enclosures can be formed by sealing (e.g., with two seals) two polymer tubes (again, the one with smaller radius positioned within the one with larger radius) and one polymer sheet which can be placed in the tube with smaller radius or between the two polymer tubes.

Further, typically, the first and second enclosure and any third, fourth or fifth enclosure can have substantially the same enclosure length. Typically, enclosure lengths can be up to several hundred meters, and enclosure width can be up to several meters. More typically, enclosure lengths are between 1 meter and 200 meters with widths between 0.5 meter and 2 meters. Even more typically, enclosure lengths are between 10 meters and 200 meters with widths between 0.5 meter and 2 meters. Yet even more typically, enclosure lengths are between 20 meters and 100 meters with widths between 0.5 meter and 2 meters.

Controllably pressuring the enclosures of the photobioreactor, particularly, the second and third enclosure, can controllably change the shape of the first enclosure. The shape of the first enclosure can be controllably changed to a shape which facilitates increased productivity of the photoautotrophic microorganisms. Alternatively, the shape of the first enclosure can be controllably changed to facilitate draining of the first reactor chamber. In another alternative, it can be controllably changed to facilitate thermal control of the culture medium. In yet another alternative, the shape of the first enclosure can be controllably changed to facility mixing of the culture medium.

The bioreactors and, particularly, photobioreactors of the present invention can be used for the production of carbon-based products of interest using photoautotroph microorganisms.

Further embodiments of the present invention are directed to methods of producing carbon based products of interest using the bioreactors and, particularly, photobioreactors as described herein. Particular carbon-based products of interest can be fuels or chemicals. Alternatively, particular carbon-based products of interest include ethanol, propanol, isopropanol, butanol, terpenes, alkanes such as pentadecane, heptadecane, octane, propane, fatty acids, fatty esters, fatty alcohols, olefins or diesel.

The enclosures as described above are typically adapted to be pressurized (i.e., inflated to a gas pressure inside the enclosure greater than the outside pressure). Because the enclosures typically have flexible walls, independently pressurizing two or more enclosures typically leads to changes to the shape of the enclosures if the pressure differences in respective enclosures are sufficiently large. Accordingly, controllably changing the pressures can controllably change one or more enclosure shapes for a given photobioreactor design.

Reactor and Support Chambers

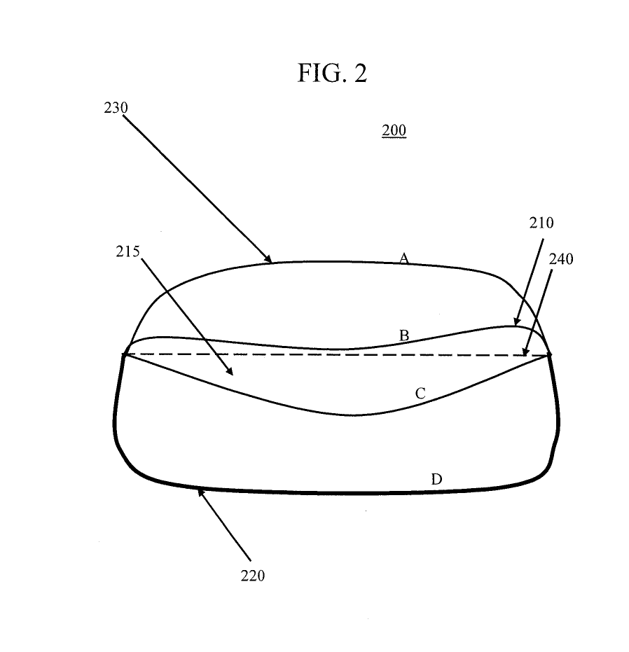

As shown in FIG. 2, a cross-section of an illustrative thin-film photobioreactor in accordance with the present invention, according to certain embodiments, is shown. The thin-film photobioreactor 200 includes reactor chambers 210 in the form of one or more channels and one or more support chambers (base chamber 220 and/or cover chamber 230). The reactor chamber 210 and support chambers 220, 230 of the photobioreactor can be adapted to allow cultivation in the culture medium of the phototrophic microorganisms 215 in a thin layer. Phototrophic organisms growing in photobioreactors can be suspended or immobilized. Typically, the layer is between about 5 mm and about 30 mm thick, or, more typically, between about 10 mm and about 15 mm. By inflating the support chambers and/or the reactor chamber 210 to predetermined amounts, the desired thickness of the layer of phototrophic microorganisms can be achieved. A substantial even layer can have a thickness with various depths ranging between about 5 mm to about 30 mm thick for a substantial surface area portion of the layer, or, more typically, between about 10 mm to about 15 mm.

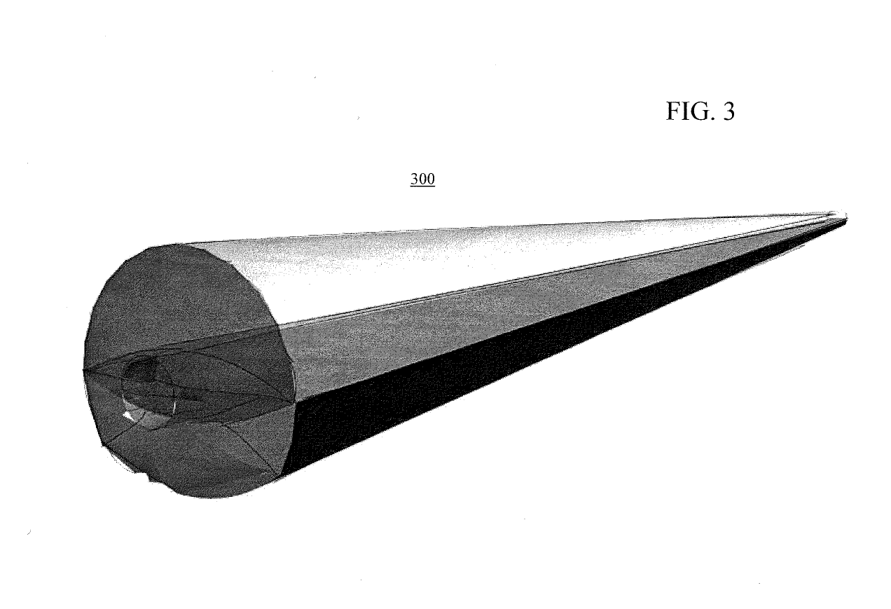

The inflating process to predetermined amounts can occur before, during, or after the introduction of the culture medium and/or microorganisms being introduced into the reactor chambers 210 and/or the inflating process can occur before, during, or after the introduction of the fill material being introduced into the support chambers 220, 230. As shown in FIG. 3, a perspective view of the thin-film photobioreactor in accordance with the present invention, according to certain embodiments, is provided. In preferred embodiments, the photobioreactor comprises a reactor chamber with one or more flexible walls for enclosing microorganisms and culture medium; and a support chamber with one or more flexible walls wherein inflating the support chamber to a predetermined amount causes the microorganisms and culture medium to distribute to a substantially even depth across the reactor chamber. A “predetermined amount” as used herein, refers to a predetermined (e.g., by calculation, experimentally, or with the use of an algorithm) pressure (e.g., a specific pressure value, or an amount of fluid) or volume. For a given bioreactor (and, particularly, photobioreactor) there can be more than one suitable amount. In further embodiments of the present invention, instead of inflating to a predetermined amount, the pressure is controllably (i.e., the pressure or pressures are changed and the effect on the reactor chamber shape and/or culture layer distribution is observed and, as necessary, further pressure changes applied based on the observed effects until a desired reactor chamber shape and/or culture layer distribution is achieved) changed to reach an amount that leads to the desired reactor chamber shape and/or culture layer distribution (typically, a thin culture layer) within the reactor chamber.

The reactor chambers 210 of the photobioreactor are shown to enclose a phototrophic microorganism and culture medium, such as algae or cyanobacteria. The reactor chamber 210 can be provided by a thin-film material enclosure, typically made from a polymeric material. The phototrophic microorganisms contained in photobioreactors for growth and/or the production of carbon-based products of interest can require light. Therefore, the photobioreactors and, in particular, the reactor chambers are adapted to provide light of a wavelength that is photosynthetically active in the phototrophic microorganism to reach the culture medium 215. Typically, at least sheets A and B of the reactor chamber 210 and cover chamber 230 can be transparent for light of a wavelength that is photosynthetically active in the phototrophic microorganism. This can be achieved by proper choice of the material, for example, thin-film material for the reactor chamber to allow light to enter the interior reactor chamber.

The base chamber 220 can be provided by a thin-film material enclosure, typically made from a polymeric material with great abrasion, and particularly, tear resistance. In applications in which the base chamber 220 can rest directly on the ground, the bottom sheet of material D can include a polymeric material of greater thickness than layers A, B and C, multiple layers, and/or additional features that can prevent abrasions or tears due to contact of courser material, such as, gravel, or other objects on the ground. These features can include, but are not limited to, impregnated mesh and/or rubberized or other protective coatings.

The photobioreactors can include one or more reactor chambers 210. The photobioreactor chambers can be of different shapes and sizes. The photobioreactor size can be influenced by the material and manufacturing choices. For example, in some embodiments of the present invention, the photobioreactors are made of a thin film polymeric material which can be, for example, between 1 and 200 meters long with a width of between about 0.5-2 meters. In some embodiments, the reactor chamber 210 is 40 meters long. A further consideration is transportability of a manufactured photobioreactor, which is greatly enhanced by using flexible thin-film. The reactor chamber 210 can be designed to be folded and/or rolled at least to some extent for more compact storage. For photobioreactors with very large reactor chambers 210 this is a significant advantage, because it can prevent costly transportation permits and oversized transport vehicles, or, alternatively, significant installation costs at the installation site.

Each reactor chamber 210 of a photobioreactor can be of a different shape and dimension. Typically, however, in photobioreactors with a plurality of reactor chambers, the reactor chambers are of similar or identical shape and dimensions, for example, channels positioned in parallel with substantially longer channel length than width. Various reactor chamber cross-sections are suitable, for example, cylindrical, or half-elliptical. Preferably, the reactor chamber is half-elliptical or rectangular. Further, reactor chamber(s) can be enclosures (e.g., bags) welded from thin polymeric films. Such reactor chambers can allow for advantageous compact transport, facilitate sterilization (e.g., with radiation such as gamma radiation) prior to deployment, and allow use as disposable reactor chamber(s) because of the cost-efficiency and/or energy efficiency of their production. They can also be reused.

The photobioreactor described herein can be placed on the ground or float on water such that the cover chamber 230 and reactor chamber 210 are directed upwards and the base chamber 220 is placed on the ground or water, respectively.

Alternatively, the photobioreactor(s) can also be placed above the ground and can utilize solid support structures made of, for example, wood, metal, mesh or fabric. Other support structures can include, for example, stakes, anchors or cables attached to flaps along the edges of the photobioreactor. In one exemplary embodiment, straps or loops are provided along both edges of the photobioreactor. The straps or loops can use stakes or anchors to secure the photobioreactor to the ground or other surface. The straps or loops can be of the same material as the base chamber 220. The straps or loops can also be of a different material and/or molded into or adhered to the multiple layers used to construct the photobioreactor. The support structures can be used to prevent rotation or movement of the photobioreactor. The support structure can be used to prevent movement due to gravity, unintended external forces and/or inclement weather.

The photobioreactors can include a number of devices that can support the operation of the photobioreactors. For example, devices for flowing gases (e.g., carbon dioxide, air, and/or other gases), measurement devices (e.g. optical density meters, thermometers), inlets and outlets, and other elements can be integrated or operationally coupled to the photobioreactor. The reactor chambers 210 can include further elements (not shown) such as inlets and outlets, for example, for growth media, carbon sources (e.g., CO₂), and probe devices such as optical density measurement device and thermometers.

Further, the photobioreactor panels can be adapted to allow gas flow through the various reactor and support chambers. Gas (e.g. CO₂) flow can be co- and/or counterdirectional to liquid flow through the reactor or support chamber(s) of the photobioreactor. For example, in certain embodiments, the photobioreactors are adapted to allow codirectional gas flow in one part of the reactor chamber and counterdirectional gas flow in another part of the reactor chamber. In other embodiments, one or more reactor chambers of a photobioreactor are adapted to allow codirectional gas flow, and one or more other reactor chambers of the photobioreactor are adapted to allow counterdirectional gas flow. The sheets of material separating the reactor and support chambers can also be designed to allow passage of gas while preventing passage of liquids, culture medium, and/or microorganisms.

In additional embodiments, support straps or a support sheet 240 can be used to further allow for a desired shape of the reactor chamber 210. Support straps 240 can be strips of polymeric material or cords spaced along the length of the chamber. The support straps or sheet 240 can also be a mesh sheet or other sheet of material with various openings. As the base chamber 220 and/or cover chamber 230 are inflated, the support straps can be brought into tension providing more of a rectangle shape with a desired thickness to the reactor chamber 210. The support straps 240 can be incorporated in the manufacturing of the photobioreactor design by placing the straps or threads between the layers B and C of thin film of the reactor chamber 210. The straps or threads can be coupled to the outer edges during an edge sealing process. The sealing process can weld or adhere the straps or threads between the various layers of the chambers. The support straps or support sheet 240 are not limited to a location within the reactor chamber 210 and can also be incorporated within either support or other chambers of the photobioreactor design.

Reactor and Support Chambers Incorporating a Thermal Chamber

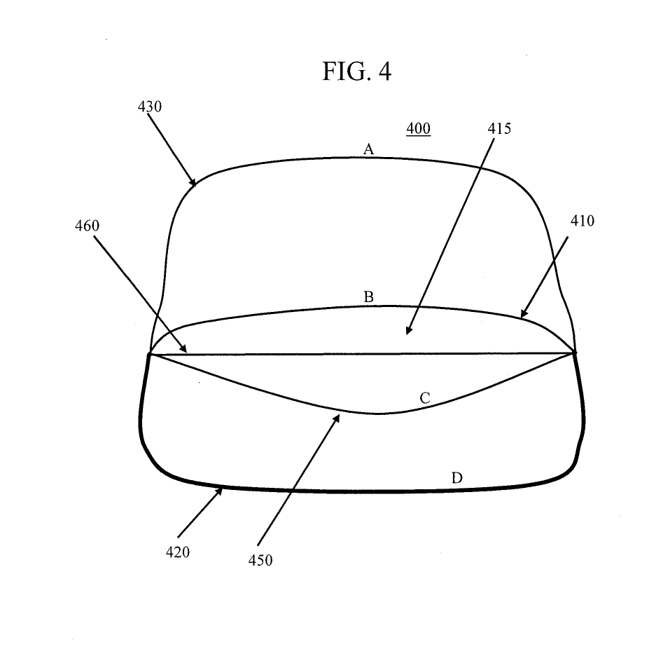

As shown in FIG. 4, a cross-section of an illustrative thin-film photobioreactor with a thermal chamber in accordance with the present invention, according to certain embodiments, is shown. The thin-film photobioreactor 400 includes reactor chambers 410 in the form of one or more channels and one or more support chambers (base chamber 420 and/or cover chamber 430). The reactor chamber 410 and support chambers of the photobioreactor can be adapted to allow cultivation in the culture medium of the phototrophic microorganisms 415 in a thin layer incorporating many aspects as previously described in the prior embodiment. Again, typically, the layer is between about 5 mm and about 30 mm thick, or, more typically, between about 10 mm and about 15 mm. By inflating the support chambers 420, 430 and/or the reactor chamber 410 to predetermined amounts, the desired thickness of the layer of phototrophic microorganisms can be achieved.

The thin-film photobioreactor 400 includes reactor chambers 410 in the form of one or more channels and can also include a corresponding heat exchange chamber 450 (bottom side) separated from the reactor chambers by a separating layer 460. Exemplary embodiments can use a similar circulation drive system to drive, for example, heat exchange liquid in the heat exchange chamber 450.

The reactor chambers 410 of the photobioreactor are shown to enclose a phototrophic microorganism and culture medium, such as algae or cyanobacteria. The reactor chamber 410 can be provided by a thin-film material enclosure, typically made from a polymeric material. The separating layer 460 can controllably separate the reactor chamber 410 from the heat exchange chamber 450, and thereby the culture medium from heat exchange liquid. In other embodiments, the heat exchange chamber 450 containing heat exchange liquid can be replaced with a solid material of high heat capacity.

The heat exchange chamber 450 as shown in this embodiment is in thermal contact with the reactor chamber through the separating layer 450. Heat exchange between the heat exchange liquid, typically water, and culture medium of the reactor chamber 410 containing phototrophic microorganisms can be established. Heat exchange between the heat exchange liquid and culture medium can be reduced significantly, for example, by reducing the heat exchange liquid level in the heat exchange chamber to form a thermally insulating gas space. In some embodiments, controlling the extent of the gas space within the heat exchange chamber 450 can control the thermal contact between the reactor chamber 410 and the heat exchange chamber 450 (an example for a heat energy system), that is, a controllable thermal contact. Other reactor chambers, systems, and methods are described in PCT publication 2011/017171, entitled: “PHOTOBIOREACTOR, SOLAR ENERGY GATHERING SYSTEMS, AND THERMAL CONTROL METHODS” filed Jul. 28, 2010, the disclosure of which is hereby incorporated by reference in their entirety.

Typically, at least sheets A and B of the reactor chamber 410 and cover chamber 430 can be transparent for light of a wavelength that is photosynthetically active in the phototrophic microorganism. Sheet C of the heat exchange chamber 450 can be made of a polymeric material and designed to support the contents of the reactor chamber 410 and heat exchange chamber 450. The base chamber 420 can be provided by a bottom sheet of material D, typically made from a polymeric material with great abrasion, and particularly, tear resistance as previously described. The photobioreactor chambers can be of different shapes and sizes to provide for the intended shape of the reactor chamber 410. Further, the reactor chamber(s) can be sheets or enclosures (e.g., tubs) welded or adhered together to provide the chambers of the photobioreactor.

The photobioreactors can include a number of devices that can support the operation of the photobioreactors. For example, devices for flowing gases (e.g., carbon dioxide, air, and/or other gases), measurement devices (e.g. optical density meters, thermometers), inlets and outlets, and other elements can be integrated or operationally coupled to the photobioreactor as previously described herein.

A heat energy system can function as a heat sink and/or heat reservoir. Typically, the heat energy system includes a material with sufficiently high heat capacity. The material can be solid, for example, a metal or polymer or liquid, preferably water. Preferably, the heat energy system includes a heat exchange chamber containing a heat exchange liquid such as water, and optionally, inlets and outlets for exchange of the heat exchange liquid.

Reactor Chambers with Capsule

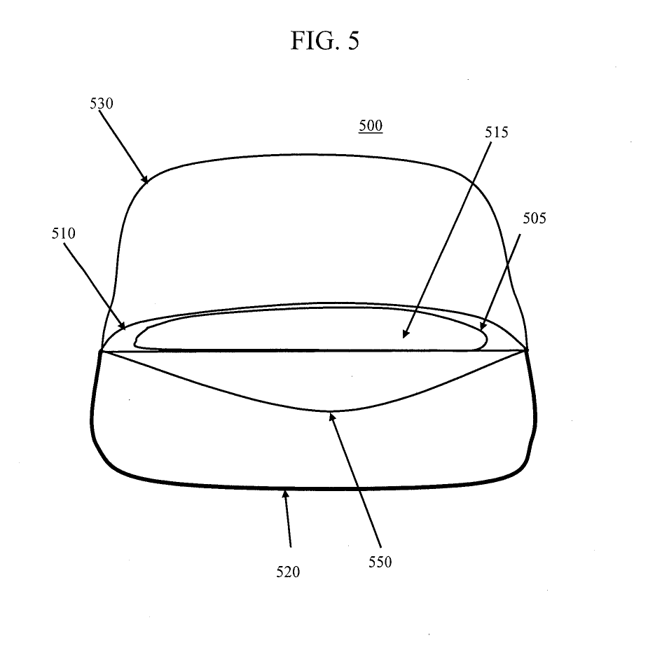

As shown in FIG. 5, a cross-section of an illustrative thin-film photobioreactor with reactor capsule in accordance with the present invention, according to certain embodiments, is shown. The thin-film photobioreactor 500 includes reactor capsule 505 housed within the reactor chamber 510 and one or more support chambers (base chamber 520 and/or cover chamber 530) and/or heat exchanger chamber 550. The reactor chamber 510 and support chambers of the photobioreactor can be adapted to allow cultivation in the culture medium of the phototrophic microorganisms 515 in a thin layer incorporating many aspects as previously described in the prior embodiment. Again, typically, the layer in the reactor capsule 505 is between about 5 mm and about 30 mm thick, or, more typically, between about 10 mm and about 15 mm. By inflating the support chambers 520, 530 and/or the reactor chamber 510 to predetermined amounts the desired thickness of the layer of phototrophic microorganisms can be achieved.

The reactor capsule 505 can provide additional protection against leakage of culture medium or phototrophic microorganisms. The reactor chamber 510 can not only be used to contain leaks but also used to indicate leaks by detecting the presence of culture medium or microorganisms within the reactor chamber 510. The inserted reactor capsule can also be designed for single use operation and manufactured in a sterile process, thus eliminating the need for cleaning between culture runs. The photobioreactor design can provide for capsules with thinner walls and reduced manufacturing costs by allow the surrounding reactor chamber 510 to surround and support the walls of the reactor capsule 505. The pressure within the reactor chamber 510 can be provided to reduce the pressure exerted on the walls of the reactor capsule 505. The reactor capsule 505 and/or culture medium and microorganisms can be inserted prior to inflation of the photobioreactor chambers to allow for easier insertion. Once inserted, the photobioreactor chambers can be inflated to provide the desired shape of the reactor capsule 505 with increased aspect ratio.

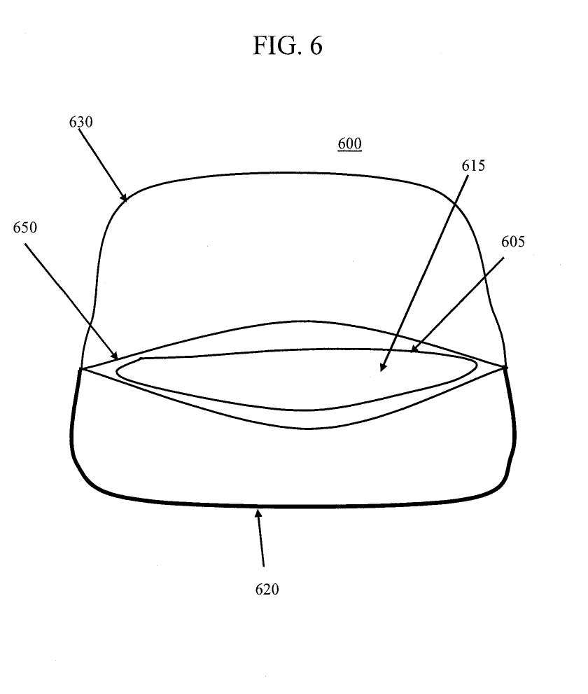

As shown in FIG. 6, a cross-section of an illustrative thin-film photobioreactor with reactor capsule in accordance with the present invention, according to certain embodiments, is shown. The thin-film photobioreactor 600 includes reactor capsule 605 housed within the heat exchanger chamber 650 and one or more support chambers (base chamber 620 and/or cover chamber 630). The heat exchanger chamber 650 holds and supports the reactor capsule 605. Similar to previous embodiments, the reactor capsule 605 can allow cultivation in the culture medium of the phototrophic microorganisms 615 in a thin layer incorporating many aspects as previously described in the prior embodiment. By inflating the support chambers 620, 630 and/or the heat exchanger chamber 650 to predetermined amounts, the desired thickness of the layer of phototrophic microorganisms can be achieved. The reactor capsule 605 can provide additional protection advantages as previously described in the previous reactor capsule embodiment of FIG. 5. Similarly, the heat exchanger chamber 650 can not only be used to contain leaks but also used to indicate leaks by detecting the presence of culture medium or microorganisms within the heat exchanger fluid.

Reactor Chamber with Crescent Shape

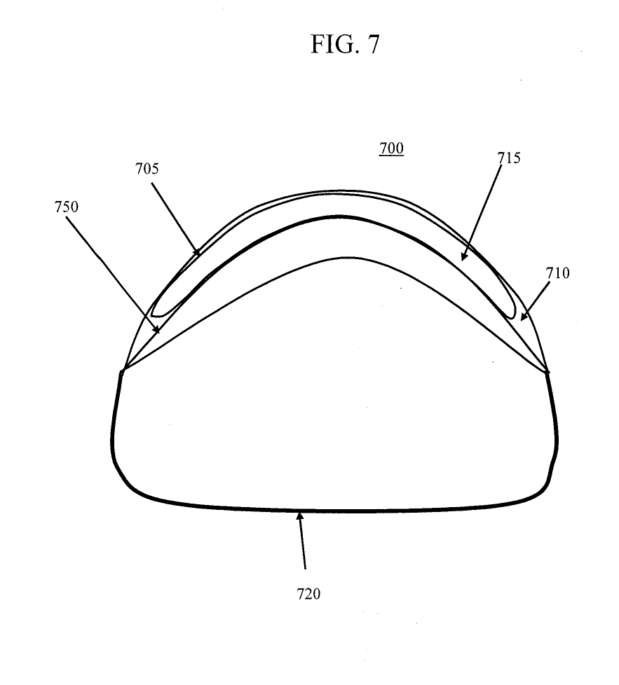

The reactor chamber can be designed to include a variety of shapes when deployed. For example, some shapes of the reactor chamber can be designed to provide better exposure to direct natural sunlight. As shown in FIG. 7, a cross-section of an illustrative thin-film photobioreactor with reactor capsule having a crescent shape in accordance with the present invention, according to certain embodiments, is shown. The thin-film photobioreactor 700 includes reactor capsule 705 housed within the reactor chambers 710 and one or more support chambers (base chamber 720) and/or heat exchanger chamber 750. The reactor chamber 710 and base chamber 720 of the photobioreactor can be adapted to allow cultivation in the culture medium of the phototrophic microorganisms 715 in a thin crescent shaped layer incorporating many aspects as previously described in the prior embodiment. Typically, the layer in the reactor capsule 705 can be between about 5 mm and about 30 mm thick, or, more typically, between about 10 mm and about 15 mm. By inflating the base chamber 720, the heat exchanger chamber 750 and/or the reactor chamber 710 to predetermined amounts, the desired thickness and crescent shape of the layer of phototrophic microorganisms can be achieved. The crescent shape of the layer can not only produce the desired aspect ratio but also can foster more direct exposure of sunlight as the sun rotates over the horizon.

Reactor and Multiple Lateral Support Chambers

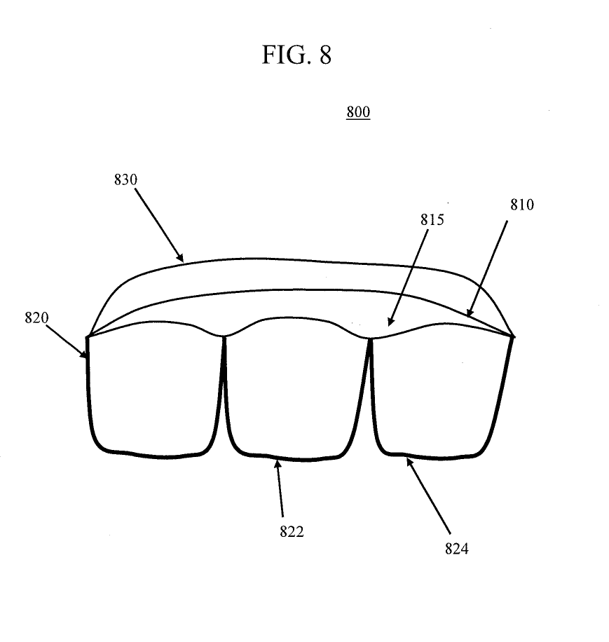

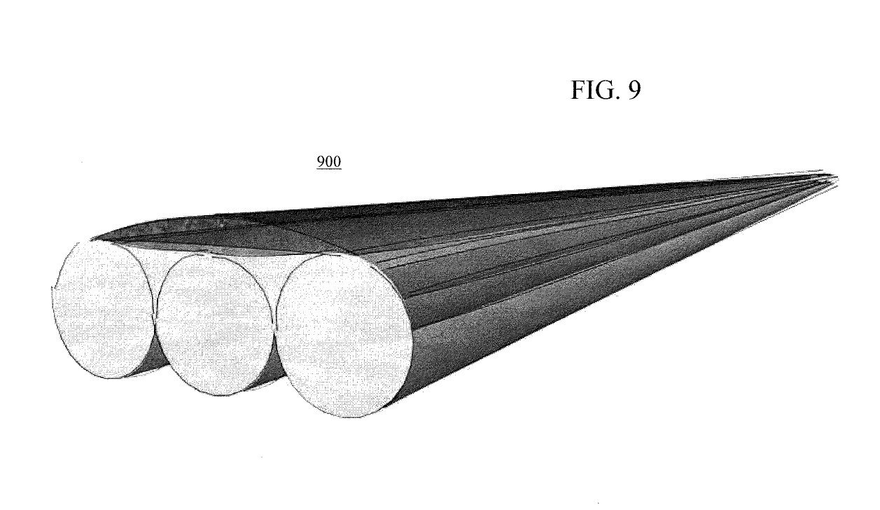

As shown in FIG. 8, a cross-section of an illustrative thin-film photobioreactor with multiple lateral support chambers in accordance with the present invention, according to certain embodiments, is shown. The thin-film photobioreactor 800 includes a reactor chamber 810 in the form of one or more channels, a cover chamber 830 and two or more support chambers 820, 822, and 824. The reactor chamber 810 and support chambers of the photobioreactor 800 can be adapted to allow cultivation in the culture medium of the phototrophic microorganisms 815 in a thin layer incorporating many aspects as previously described in the prior embodiment. Again, typically, the layer is between about 5 mm and about 30 mm thick, or, more typically, between about 10 mm and about 15 mm. By inflating the support chambers 820, 822, and 824 and/or the reactor chamber 810 and cover chamber 830 to predetermined amounts, the desired shape and thickness of the layer of phototrophic microorganisms can be achieved. As shown in FIG. 9, a perspective view of the thin-film photobioreactor with multiple lateral support chambers in accordance with the present invention, according to certain embodiments, is provided.

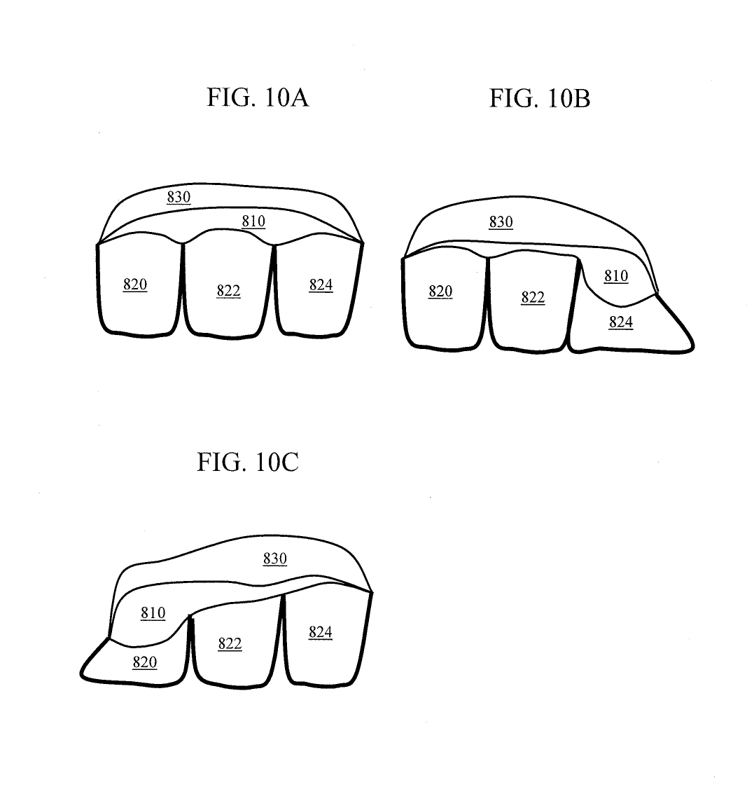

Referring to FIGS. 10A-C, the multiple lateral support chambers 820, 822, and 824 can provide additional advantages by allowing mixing of culture medium or phototrophic microorganisms. By deflating and/or inflating the various lateral support chambers 820, 822, and 824, the culture medium and phototrophic microorganisms can be mixed producing a side-to-side motion within the reactor chamber 810. This side-to-side action can better foster mixing not only in the direction of circulation but in a more up and down vertical direction. The process can continuously rotate in a sequence of deflating and inflating lateral support chambers 820, 822, and 824 in a series. Not only can the process provide the advantage of mixing the culture medium and phototrophic microorganisms, the process can also enhance the thermal exchange between the reactor chamber 810 and a heat exchange chamber (not shown but as described in previous embodiments).

Reactor and Multiple Segmented Support Chambers

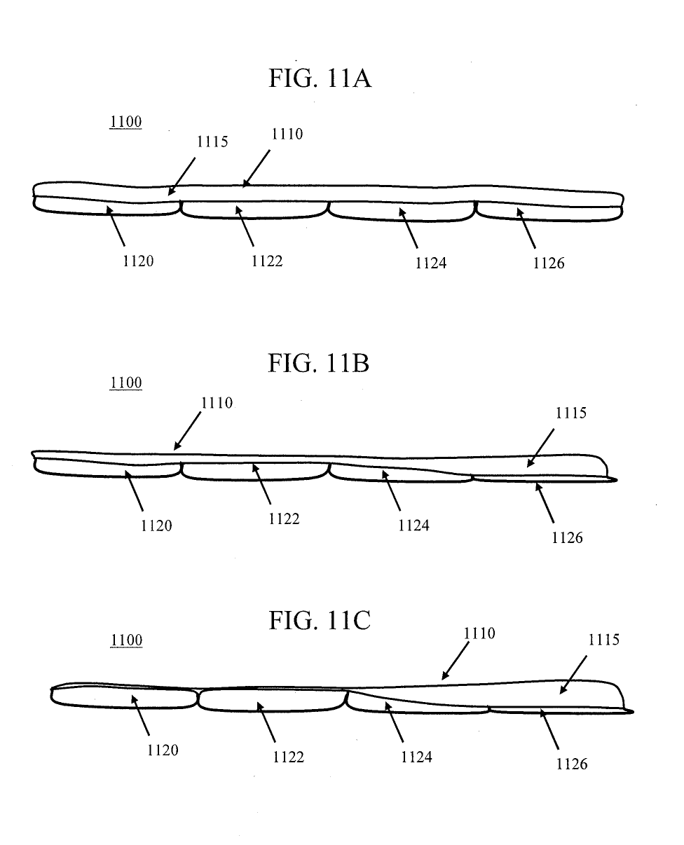

Referring to FIGS. 11A-C, a lengthwise cross-section of an illustrative thin-film photobioreactor with multiple lateral support segments in accordance with the present invention, according to certain embodiments, is shown. The thin-film photobioreactor 1100 includes reactor chambers 1110 in the form of one or more channels and two or more support chambers in segments 1120, 1122, 1124, and 1126. Embodiments of the photobioreactor 1100 can also include a cover chamber (not shown). The reactor chamber 1110 and support chambers of the photobioreactor 1100 can be adapted to allow cultivation in the culture medium of the phototrophic microorganisms 1115 in a thin layer incorporating many aspects as previously described in the prior embodiment. Again, typically, the layer is between about 5 mm and about 30 mm thick, or, more typically, between about 10 mm and about 15 mm. By inflating the support chambers and/or the reactor chamber 1110 to predetermined amounts, the desired shape and thickness of the layer of phototrophic microorganisms can be achieved. The multiple segmented support chambers 1120, 1122, 1124, and 1126 can provide additional advantages by allowing draining of culture medium or phototrophic microorganisms in addition to mixing. By deflating and/or inflating the various segments of the support chambers 1120, 1122, 1124, and 1126, the culture medium and phototrophic microorganisms can be mixed within the reactor chamber 110. Again, the process can continuously rotate in a sequence of deflating and inflating the segments of the support chambers 1120, 1122, 1124, and 1126 in a series.

Additionally, a sequential process of deflating the segments of the support chambers 1120, 1122, 1124, and 1126 one after another can be used to direct the flow of culture medium and microorganisms towards a drain in the reactor chamber 1110. Alternatively, the process can be used to direct the flow away from an entrance port during a process of filling the reactor chamber 1110 with culture medium and microorganisms.

Support Structures

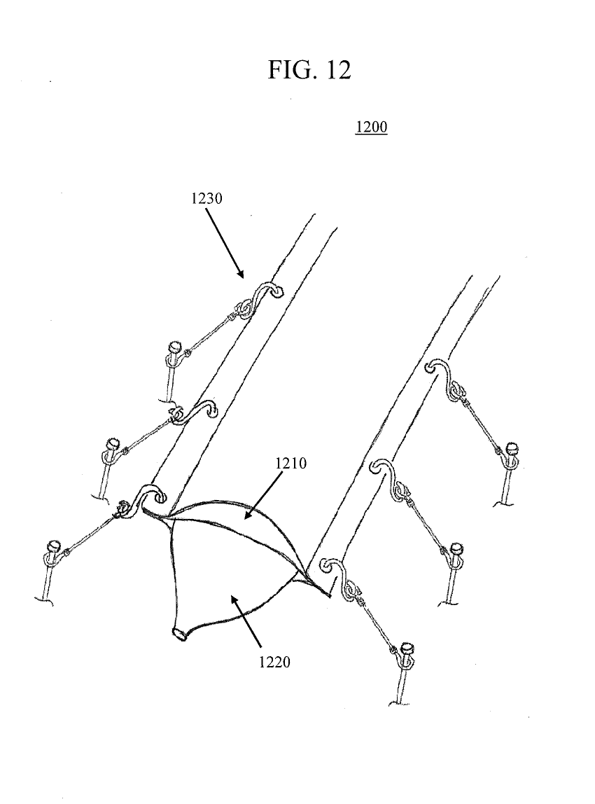

Referring to FIG. 12, the photobioreactor 1200 includes support structures 1230 along both edges of the photobioreactor 1200. The support structures 1230 can be straps or loops used to stake or anchor the photobioreactor 1210 to the ground or other surface. The straps or loops can be of the same material as the reactor chamber 1210 the base chamber 1220. The straps or loops can also be of a different material and/or molded into or adhered to the multiple layers used to construct the photobioreactor 1200. The support structures 1210 can be used to prevent rotation or movement of the photobioreactor 1200. The support structure 1210 can be used to prevent movement due to gravity, unintended external forces and/or inclement weather.

Photobioreactor Biomass Productivity

The solar biofactory also provides methods to achieve organism productivity as measured by production of desired products, which includes cells themselves.

The desired level of products produced from the engineered light capturing organisms in the solar biofactory system can be of commercial utility. For example, the engineered light capturing organisms in the solar biofactory system convert light, water and carbon dioxide to produce fuels, biofuels, biomass or chemicals at about 5 to about 10 g/m²/day, in certain embodiments about 15 to about 42 g/m²/day and in more preferred embodiments, about 30 to 45 g/m²/day or greater.

The photobioreactor system affords high areal productivities that offset associated capital cost. Superior areal productivities are achieved by: optimizing cell culture density through control of growth environment, optimizing CO₂ infusion rate and mass transfer, optimizing mixing to achieve highest photosynthetic efficiency/organisms, achieving maximum extinction of insolating light via organism absorption, and achieving maximum extinction of C0₂ and initial product separation.

In particular, the southwestern U.S. has sufficient solar insolation to drive maximum areal productivities to achieve about >25,000 gal/acre/year ethanol or about >15,000 gal/acre/year diesel, although a majority of the U.S. has insulation rates amenable to cost effective production of commodity fuels or high value chemicals.

Furthermore, CO₂ is also readily available in the southwestern U.S. region, which is calculated to support large scale commercial deployment of the invention to produce 25-70 g/m²/day ethanol, or 70 Bn gal/year diesel.

### DEFINITIONS

“Carbon-based products of interest” include alcohols such as ethanol, propanol, isopropanol, butanol, fatty alcohols, fatty acid esters, ethyl esters, wax esters; hydrocarbons and alkanes such as pentadecane, heptadecane, propane, octane, diesel, Jet Propellant 8 (JP8); polymers such as terephthalate, 1,3-propanediol, 1,4-butanediol, polyols, Polyhydroxyalkanoates (PHA), poly-beta-hydroxybutyrate (PHB), acrylate, adipic acid, ε-caprolactone, isoprene, caprolactam, rubber; commodity chemicals such as lactate, docosahexaenoic acid (DHA), 3-hydroxypropionate, γ-valerolactone, lysine, serine, aspartate, aspartic acid, sorbitol, ascorbate, ascorbic acid, isopentenol, lanosterol, omega-3 DHA, lycopene, itaconate, 1,3-butadiene, ethylene, propylene, succinate, citrate, citric acid, glutamate, malate, 3-hydroxypropionic acid (HPA), lactic acid, THF, gamma butyrolactone, pyrrolidones, hydroxybutyrate, glutamic acid, levulinic acid, acrylic acid, malonic acid; specialty chemicals such as carotenoids, isoprenoids, itaconic acid; pharmaceuticals and pharmaceutical intermediates such as 7-amino deacetoxycephalosporanic acid (7-ADCA)/cephalosporin, erythromycin, polyketides, statins, paclitaxel, docetaxel, terpenes, peptides, steroids, omega fatty acids and other such suitable products of interest. Such products are useful in the context of biofuels, industrial and specialty chemicals, as intermediates used to make additional products, such as nutritional supplements, neutraceuticals, polymers, paraffin replacements, personal care products and pharmaceuticals.

As used herein, “light of a wavelength that is photosynthetically active in the phototrophic microorganism” refers to light that can be utilitzed by the microorganism to grow and/or produce carbon-based products of interest, for example, fuels including biofuels.

As used herein, “flexible wall” refers to a sheet or sheets of material that have the ability to flex or bend under a relative force or pressure is applied to a surface during operation.

“Phototrophs” or “photoautotrophs” are organisms that carry out photosynthesis such as, eukaryotic plants, algae, protists and prokaryotic cyanobacteria, green-sulfur bacteria, green non-sulfur bacteria, purple sulfur bacteria, and purple non-sulfur bacteria. Phototrophs include natural and engineered organisms that carry out photosynthesis and hyperlight capturing organisms.

The photobioreactors of the present invention are adapted to support a biologically active environment that allows chemical processes involving photosynthesis in organisms such as phototrophic organisms to be carried out, or biochemically active substances to be derived from such organisms. The photobioreactors can support aerobic or anaerobic organisms.

As used herein, “organisms” encompasses autotrophs, phototrophs, heterotrophs, engineered light capturing organisms and at the cellular level, e.g., unicellular and multicellular.

A “spectrum of electromagnetic radiation” as used herein, refers to electromagnetic radiation of a plurality of wavelengths, typically including wavelengths in the infrared, visible and/or ultraviolet light. The electromagnetic radiation spectrum is provided by an electromagnetic radiation source that provides suitable energy within the ultraviolet, visible, and infrared, typically, the sun.

A “biosynthetic pathway” or “metabolic pathway” refers to a set of anabolic or catabolic biochemical reactions for converting (transmuting) one chemical species into another. For example, a hydrocarbon biosynthetic pathway refers to the set of biochemical reactions that convert inputs and/or metabolites to hydrocarbon product-like intermediates and then to hydrocarbons or hydrocarbon products.

Anabolic pathways involve constructing a larger molecule from smaller molecules, a process requiring energy. Catabolic pathways involve breaking down of larger molecules, often releasing energy.

As used herein, “light” generally refers to sunlight but can be solar or from artificial sources including incandescent lights, LEDs fiber optics, metal halide, neon, halogen and fluorescent lights.

Throughout this specification and claims, the word “comprise” or variations such as “comprises” or “comprising,” will be understood to imply the inclusion of a stated integer or group of integers but not the exclusion of any other integer or group of integers.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of this invention are presented for purposes of illustration and description. They are not intended to be exhaustive or to limit the invention to the precise forms disclosed; obviously many modifications and variations are possible in view of the above teachings. The embodiments were chosen and described in order to best explain the principles of the invention and its practical applications. These procedures will enable others, skilled in the art, to best utilize the invention and various embodiments with various modifications. It is intended that the scope of the invention be defined by the following claims and their equivalents. Modifications and substitutions by one of ordinary skill in the art are considered to be within the scope of the present invention, which is not to be limited except by the following claims.

While this invention has been particularly shown and described with references to example embodiments thereof, it will be understood by those skilled in the art that various changes in form and details can be made therein without departing from the scope of the invention encompassed by the appended claims.

The invention claimed is:

1. A bioreactor comprising:
a reactor chamber with one or more flexible walls for enclosing microorganisms and culture medium; and
a support chamber with one or more flexible walls wherein inflating the support chamber to a predetermined amount causes the microorganisms and culture medium to distribute to a substantially even depth across the reactor chamber.

2. The bioreactor of claim 1, wherein the substantially even depth across the reactor chamber averages between about 5 mm to about 30 mm.

3. The bioreactor of claim 1, wherein the substantially even depth across the reactor chamber ranges between depths of about 5 mm to about 30 mm.

4. The bioreactor of claim 1, wherein the predetermined amount causes the microorganisms and culture medium to distribute to a substantially even depth across the reactor chamber in a convex, crescent shape.

5. The bioreactor of claim 1, wherein the support chamber is a base chamber positioned underneath the reactor chamber.

6. The bioreactor of claim 5, wherein the base chamber is an abrasion and/or tear resistant material.

7. The bioreactor of claim 1, wherein the support chamber is a cover chamber positioned overtop the reactor chamber.

8. The bioreactor of claim 7, wherein the cover chamber is a UV stable material.

9. The bioreactor of claim 7, wherein the cover chamber is transparent for light of a wavelength that is photosynthetically active to the microorganisms.

10. The bioreactor of claim 1, further comprising a thermal chamber in thermal contact with the reactor chamber.

11. The bioreactor of claim 10, wherein the thermal chamber is housed within the support chamber.

12. The bioreactor of claim 10, wherein the thermal chamber is housed within the reactor chamber.

13. The bioreactor of claim 1, wherein a reactor capsule encloses the microorganisms and the culture medium and the reactor capsule is housed within the reactor chamber.

14. The bioreactor of claim 1, wherein inflating the support chamber to a predetermined amount causes the microorganisms and culture medium to evacuate the reactor chamber.

15. The bioreactor of claim 1, wherein the support chamber comprises multiple chambers.

16. The bioreactor of claim 15, wherein inflating each multiple chamber a predetermined amount causes the microorganisms and culture medium to distribute to a substantially even depth across the reactor chamber.

17. The bioreactor of claim 15, wherein inflating one or more of each multiple chamber induces mixing the microorganisms and culture medium within the reactor chamber.

18. The bioreactor of claim 1, wherein the reactor chamber is a thin-wall construction that temporally couples to a circulation driver producing a flow of the microorganisms and culture medium within the reactor chamber.

19. The bioreactor of claim 1, wherein a first sheet, a second sheet, and a third sheet are connected lengthwise along opposing ends and the first sheet and the second sheet produce the flexible walls of the reactor chamber and the second sheet and the third sheet produce the flexible walls of the support chamber.

20. The bioreactor of claim 1, further comprising:
support structures coupled to each lengthwise edge of the photobioreactor.

21. A bioreactor comprising:
a reactor chamber with flexible, thin top and bottom walls for enclosing microorganisms and culture medium;
a base chamber with a flexible, abrasion and/or tear resistant, bottom wall for contacting the ground; and
a cover chamber with a flexible top wall transparent to light of a wavelength that is photosynthetically active to the microorganisms wherein inflating the base chamber and/or cover chamber to a predetermined amount(s) causes the microorganisms and culture medium to distribute to a substantially even depth across the reactor chamber.

22. The bioreactor of claim 21, wherein the wall of the cover chamber connects lengthwise along opposing ends to the top wall of the reactor chamber and the wall of the base chamber connects lengthwise along opposing ends to the bottom wall of the reactor chamber.

23. The bioreactor of claim 21, further comprising a thermal chamber with a flexible, thin wall wherein the wall of the cover chamber connects lengthwise along opposing ends to the top wall of the reactor chamber, the wall of the thermal chamber connects lengthwise along opposing ends to the bottom wall of the reactor chamber, and the wall of the base chamber connects lengthwise along opposing ends to the wall of the thermal chamber.

24. A method of culturing phototrophic microorganism in the photobioreactor comprising the actions of:
inflating a support chamber to a predetermined amount causing the microorganisms and culture medium to distribute to a substantially even depth across a thin film reactor chamber; and
circulating the microorganisms and culture medium through the thin film reactor chamber.

25. The method of culturing phototrophic microorganism in the photobioreactor of claim 24,
wherein the support chamber is transparent to light of a wavelength that is photosynthetically active to the microorganisms.

26. The method of culturing phototrophic microorganism in the photobioreactor of claim 24, further comprising the action of:
circulating a fluid in a thermal chamber in thermal contact with the reactor chamber.

27. The method of culturing phototrophic microorganism in the photobioreactor of claim 24, further comprising the action of:
inserting a reactor capsule for enclosing the microorganisms and the culture medium within the reactor chamber prior to the action of circulating the microorganisms and culture medium.

28. The method of culturing phototrophic microorganism in the photobioreactor of claim 24, further comprising the action of:
inflating the support chamber to a second predetermined amount causes the microorganisms and culture medium to evacuate the reactor chamber.

29. The method of culturing phototrophic microorganism in the photobioreactor of claim 24, wherein the support chamber comprises multiple support chambers and each is inflated to a predetermined amount causing the microorganisms and culture medium to distribute to the substantially even depth across the thin film reactor chamber.

30. The method of culturing phototrophic microorganism in the photobioreactor of claim 29, wherein inflating one or more of each multiple chamber induces mixing the microorganisms and culture medium within the reactor chamber.

31. A photobioreactor comprising:
a reactor chamber with one or more flexible walls for enclosing microorganisms and culture medium; and
a support chamber with one or more flexible walls wherein inflating the support chamber to a predetermined amount causes a surface area of the microorganisms and culture medium to increase.

32. The photobioreactor of claim 31, wherein inflating the support chamber to a predetermined amount causes a surface area of the microorganisms and culture medium to increase and the depth of the microorganisms and culture medium to decrease.

33. The photobioreactor of claim 32, wherein the substantially even depth of the microorganisms and culture medium across the reactor chamber averages between about 10 mm to about 15 mm.

34. The photobioreactor of claim 32, wherein the substantially even depth of the microorganisms and culture medium across the reactor chamber ranges between about 10 mm to about 15 mm.

35. The photobioreactor of claim 32, wherein the substantially even depth of the microorganisms and culture medium across the reactor chamber is across a width or a length of the reactor chamber.

36. A photobioreactor comprising: a first enclosure for containing a culture medium with a photoautotrophic microorganism and a second enclosure; wherein (1) the first enclosure and the second enclosure are layered with the second enclosure being below the first enclosure, (2) the first enclosure having at least a flexible bottom section and the second enclosure having at least a flexible top section, (3) the first enclosure and second enclosure are positioned such that controllably pressurizing the second enclosure controllably changes the shape of the first enclosure containing a culture medium with a phototrophic microorganism and a gas, and (4) the first enclosure being at least in part transparent to allow light of a wavelength that is photosynthetically active in the phototrophic microorganism to reach the culture medium.

37. The photobioreactor of claim 36, further comprising a third enclosure having at least a flexible bottom and/or top section, wherein (4) the third enclosure is layered above the second enclosure, and the third enclosure is (a) layered above the first enclosure or (b) encloses the first enclosure, (5) the first enclosure, second enclosure and third enclosure are positioned such that controllably pressurizing the second and third enclosure controllably changes the shape of the first enclosure containing a culture medium with a phototrophic microorganism and a gas, and (6) the third enclosure being at least in part transparent to allow light of a wavelength that is photosynthetically active in the phototrophic microorganism to reach the culture medium.

38. The photobioreactor of claim 37, further comprising a fourth enclosure having at least a flexible top section, wherein (7) the fourth enclosure is layered above the second enclosure, the fourth enclosure is layered below the third enclosure, and the fourth enclosure is (a) layered below the first enclosure or (b) encloses the first enclosure, provided that when the first enclosure is enclosed by the third enclosure, the fourth enclosure is below the first enclosure, (8) the fourth enclosure being positioned and adapted to allow substantial heat exchange between liquid contained therein with the culture medium.

39. The photobioreactor of any one of claims 36 to 38, wherein the culture medium and gas are contained within a fifth enclosure which is contained within the first enclosure, wherein the fifth enclosure is (9) at least in part transparent to allow light of a wavelength that is photosynthetically active in the phototrophic microorganism to reach the culture medium.

40. The photobioreactor of any one of claims 36 to 39, wherein the first, second, third, and/or fourth enclosure are formed by lengthwise sealing of polymer sheets.

41. A method of producing a carbon-based product using a photobioreactor of any one of claims 36 to 40, the method comprising:
circulating culture medium including a phototrophic microorganism through the first enclosure; and
contacting the culture medium with carbon dioxide under conditions suitable for the phototrophic microorganism to produce the carbon-based product.

42. The method of claim 41, further comprising:
flowing a fluid through a thermal chamber in thermal contact with the first enclosure.

43. The method of claim 42, wherein the thermal chamber is provided by the fourth enclosure.

44. The method of any one of claims 41 to 43, further comprising:
controllably changing the shape of the first enclosure by controllably pressurizing the second enclosure and optionally one or more of the other enclosures to cause the culture medium to be substantially removed from the first enclosure.

45. The method of any one of claims 41 to 44, further comprising:
controllably changing the shape of the first enclosure by controllably pressurizing the second enclosure and optionally one or more of the other enclosures to induce mixing of the microorganisms and culture medium within the first enclosure.
